(19) United States
(12) Patent Application Publication
(10) Pub. No.: US 2010/0146644 A1
(43) Pub. Date: Jun. 10, 2010

(54) TRANSGENIC NON-HUMAN ANIMAL

(76) Inventor: Roland John Mayer, Nottingham (GB)

Correspondence Address: FENWICK & WEST LLP, SILICON VALLEY CENTER, 801 CALIFORNIA STREET, MOUNTAIN VIEW, CA, 94041, US

(21) Appl. No.: 12/513,661

(22) PCT Filed: Oct. 31, 2007
(86) PCT No.: PCT/GB2007/004161

§ 371 Date: Jan. 27, 2010

(30) Foreign Application Priority Data
Nov. 7, 2006 (GB) 0622145.1

(51) Int. Cl. G01N 33/50; A01K 67/027; C12N 15/01

(52) U.S. Cl. 800/3; 800/9; 800/21

(57) ABSTRACT

A transgenic non-human animal genetically modified to have a dysfunctional 26S-proteasome in some or all cells, which may exhibit phenotypic and/or neuropathological symptoms similar to those exhibited by individuals with a neurogenerative disorder.

The present invention relates to transgenic non-human animal models for studying disease, and in particular to models for studying neurodegenerative diseases such as Parkinson's disease or dementia with Lewy bodies.

Parkinson's disease and dementia with Lewy bodies are among the most commonly found neurodegenerative disorders in the elderly. Dementia with Lewy bodies (DLB) is found in 15-30% of patients with irreversible cognitive decline (Weiner, M. F. Archives of Neurology (1999) 56, 1441-1442). Intraneuronal Lewy bodies are found alone in DLB but 50-60% of patients with Alzheimer's disease have cortical Lewy bodies as well as neurofibrillary tangles (Hamilton, R. L. Brain Pathol (2000) 10, 378-84).

Idiopathic Parkinson's disease is the second most common cause of chronic neurodegenerative disease with incidence increasing from approximately 40 cases per 100000 individuals to 110 per 100000 in the age range of 60-69 to 70-99 years (Van Den Eeden, S. K. et al. Am J Epidemiol (2003) 157, 1015-22). The disease is characterised by progressive loss of dopaminergic neurones in the nigrostriatal pathway in the brain with ensuing motor dysfunction. Although several drugs, including L-Dopa, are efficacious in the disease neuronal loss continues to occur. The fundamental mechanisms of pathogenesis are not understood although mitochondrial dysfunction and the generation of reactive oxygen species have been implicated in neuronal loss and disease progression. The central role of mitochondria in disease pathogenesis was demonstrated recently by evidence from Drosophila that the PTEN-induced kinase 1 (PINK1) and the ubiquitin protein ligase parkin, both of which are mutated in familial Parkinson's disease, are needed for normal mitochondrial activities and appear to operate in the same biochemical pathway (Park, J. et al. Nature (2006) 441, 1157-1161 and Clark, I. E. et al. Nature (2006) 441, 1162-1166).

There are major findings that indicate that the ubiquitin proteasome system (26S-proteasome) is involved in the pathogenesis of neurodegenerative diseases and the formation of cortical Lewy bodies (Lowe et al. J Pathol (1988) 155, 9-15; Minichiello et al. Neuron (1999) 24, 401-414; Ciechanover et al. Neuron (2003) 40, 427-445 and Cruts et al. Nature (2006) 442, 920-924).

Attempts to establish animal models to study degenerative neurological disorders have been disappointing. Most models have relied on the overexpression of particular proteins, for example, over expression of alpha synuclein which is found in Lewy bodies and/or overexpression of tau which is found in neurofibrillary tangles (McGowan et al. Trends in Genetics (2006) 22, 281-289). Over expression of protein is not believed to be the cause of the diseases, and although animals with symptoms and pathologies similar to the human condition can be generated this way the cause is different and thus the animals are not good models for developing drugs or understanding the pathways behind the diseases.

The aim of this invention is to provide an improved animal model for use in studying neurodegenerative disorders.

To date there is no available model organism in which to study Parkinson's disease and/or dementia with Lewy bodies in which the function of the 26S-proteasome has been disrupted.

The 26S-proteasome comprises a barrel-shaped multi-protein complex which can specifically digest other proteins into short polypeptides and amino acids in an ATP driven reaction. Essentially, unwanted and misfolded proteins are ubiquitinylated in the cell and then recognised by the 26S proteasome and digested.

According to a first aspect, the present invention provides a transgenic non-human animal genetically modified to have a dysfunctional 26S-proteasome in some or all cells.

Dysfunction of the 26S-proteasome refers to a reduction or loss in activity of the 26S proteasome compared to levels of activity in a normal animal in a disease free state. Preferably, the reduction or loss in activity occurs due to a reduction in the normal or naturally occurring levels of the 26S-proteasome.

Preferably the non-human animal is a mammal, more preferably a rodent, preferably a mouse or a rat.

The term “genetically modified” refers to any purposeful alteration of the naturally occurring genome of an animal. For example, sequences may have been inserted or removed from the genome of the animal. Sequences may have been inserted into the genome which, when activated, can cause the selective deactivation of a particular gene, but when not activated have no effect on expression of the gene. Preferably, the inserted sequences are located adjacent to or within the gene to be deactivated.

The term “transgenic animal” refers to an animal which has been genetically modified. Preferably a transgenic animal includes a transgene that is inserted into the genome of one or more cells in the animal. The transgene may be integrated into the genome of somatic and/or germ line cells. A “transgene” is a piece of DNA which is partly or totally heterologous to the animal in which it is found, or is homologous to the animal in which it is found but has been inserted in the animal's genome at a non-natural position. A transgenic animal may include one or more transgenes. A transgene does not need to encode a protein; it may simply serve to disrupt gene expression or to act as a recognition site for an enzyme which may disrupt gene expression.

Preferably dysfunction of the 26S-proteasome arises due to a dysfunction in one or more of the ATPases of the 19S regulator of the 26S-proteasome.

Preferably, one or more of the ATPases of the 19S regulator of the 26S-proteasome are genetically modified.

Preferably the transgenic animal does not display over-expression of one or more of the ATPases of the 19S regulator in the 26S-proteasome. Most known model organisms for neurodegenerative diseases rely on the over expression of a particular gene product in order to produce a disease state in the organism, this is not reflective of real life disease states in humans where neurodegenerative diseases are generally not believed to be caused by over expression of a particular gene. With the exception of Down's patients which become demented which have an extra copy of the APP gene on chromosome 21, and some families with dementia with Lewy bodies have been found to have an extra copy of the α-synuclein gene.

Preferably the one or more ATPases, which are genetically modified to be dysfunctional in a transgenic animal according to the invention, are selected from the 26S-proteasome ATPases S4, S6a, S6b, S7, S8 and S10b. More preferably, the ATPase which is genetically modified is the S4 ATPase of the 19S regulator of the 26S-proteasome.

If the activity of one of the ATPases in the 26S-proteasome is disrupted then the ubiquitin proteasome pathway will not function properly. The S4 ATPase is a particularly good choice for modification as it is believed that its position in the 19S regulator of the 26S-proteasome is such that it opens the end of the proteasome to allow protein to enter the proteasome. The dysfunctioning of the S4 ATPase is therefore detrimental to the functioning of the proteasome (Rubin et al. EMBO J (1998) 17, 4909-4919).

Preferably the transgenic animal is modified such that dysfunction in the 26S-proteasome is selective, that is, the dysfunction is only seen in some cells in the transgenic animal. Alternatively, the 26S-proteasome may be dysfunctional in substantially all cells in the transgenic animal. Preferably, if one or more ATPases in the 19S regulator of the 26S-proteasome are dysfunctional, they are dysfunctional in only some cells in the transgenic animal. By controlling where the dysfunction in the 26S-proteasome occurs, different phenotypes in the transgenic animal may be observed.

By causing dysfunction of 26S-proteasomes in the anterior forebrain, transgenic non-human animals with a phenotype similar to dementia with Lewy bodies is observed. By causing dysfunction of 26S-proteasomes in catecholamine neurones, including dopaminergic neurones in the substantia nigra, transgenic non-human animals with a phenotype similar to Parkinson's disease is observed.

Preferably all or part of one or more of the genes encoding one or more of the ATPases of the 19S regulator of the 26S-proteasome are “floxed” in a transgenic animal according to the invention. By floxing all or part of the genes encoding one or more of the ATPases of the 26S-proteasome, selective dysfunction of the floxed genes can be effected by controlling where the Cre enzyme is expressed in the transgenic animal.

Alternatively, other genes important in the 26S-proteasome may be floxed.

The Cre-loxP system, which involves the insertion of loxP sites in genomic DNA and expression of the Cre recombinase enzyme is a well known technique, and would be readily understood by the man skilled in the art (Kos, C. H. Nutrition Reviews (2004) 62, 243-246)

When a gene is floxed it means that loxP sites have been introduced into the genome in or around the gene of interest, which in the presence of the Cre enzyme will allow for recombination to occur causing the deletion or inversion of the genomic DNA between the loxP sites. In the case of transgenic animals according to this invention the loxP sites may flank all or part of one or more of the ATPase genes of the 19S regulator of the 26S-proteasome, such that in the presence of the Cre enzyme, activity of the one or more ATPases is reduced or eliminated. Preferably, expression of the floxed genes is unaffected in the absence of the Cre enzyme. The loxP sites may be located in non-coding intron sequences.

Transgenic animals may be genetically modified to express the Cre enzyme, alternatively the Cre enzyme may be introduced by other means into animals with floxed genes. The Cre enzyme may be introduced into a transgenic animal by injection and/or by administration of a virus. Expression of the Cre enzyme may be arranged to occur in all cells in the transgenic animal, or may be controlled to be localised to a particular area and/or type of cell in the transgenic animal.

By controlling where the Cre enzyme is expressed, in a transgenic animal in which one or more of the genes encoding proteins (such as ATPases) of the 26S-proteasome have floxed regions, dysfunction of the 26S-proteasome can be confined to specific cells types.

According to a preferred embodiment, a transgenic animal according to the invention comprises a genome in which part or all of the genes encoding one or more of the ATPases of the 26S-proteasome have been floxed, such that in the presence of the Cre enzyme the activity of the floxed gene is reduced or eliminated, thereby causing a dysfunction in the activity of the 26S-proteasome.

Preferably transgenic animals according to the invention exhibit phenotypic (behavioural) and/or neuropathological symptoms similar to those exhibited by individuals with neurodegenerative disorders, such as Parkinson's disease and dementia with Lewy bodies, making the mice useful model systems for screening for drugs that ameliorate the symptoms of such neurodegenerative diseases. The animals may also be used to test gene or cell therapies for neurodegenerative diseases.

Preferably the loss of, or reduction of activity of, the 26S-proteasome is confined to localised regions of the brain in the transgenic animal. By controlling where there is a loss or reduction in 26S-proteasome activity the characteristics of the transgenic animal can be controlled. This allows animals with symptoms of Parkinson's disease and animals with symptoms of dementia with Lewy bodies to be produced by controlling where in the animal a loss or reduction in 26S-proteasome activity occurs.

By preventing or reducing 26S-proteasome associated ATPase activity in the anterior forebrain of animals, such as mice, the neuropathology of dementia with Lewy bodies is produced with behavioural abnormalities and memory deficits corresponding to dementia with Lewy bodies. The neuropathological features include the occurrence of abundant alpha synuclein and ubiquitin-containing cortical Lewy bodies. These pathological changes are accompanied by extensive neuronal loss with many apoptotic neurones, including neuronal dark bodies.

By preventing or reducing 26S-proteasome associated ATPase activity in catecholaminergic neurones, including dopaminergic neurones, in the brain of an animal, such as a mouse, the neuropathological features of Parkinson's disease are produced, including neuronal loss in the substantia nigra. The neuropathological features include the occurrence of abundant alpha synuclein, P62 and ubiquitin-containing Lewy bodies in the substantia nigra and Lewy neurites containing ubiquitylated protein emanating from any remaining tyrosine hydroxylase-positive neurones. Corresponding deficits in brain catecholamines are also observed.

One way to control where dysfunction of the 26S-proteasome occurs is to use the Cre-lox system, and to link expression of the Cre enzyme to a protein expressed in only some cells of the animal. Dysfunction of the 26S-proteasome will only be seen in cells where the Cre enzyme is expressed. For example, by linking Cre enzyme expression to tyrosine hydroxylase expression, expression of the Cre enzyme, and thus dysfunction of any floxed genes, is restricted to the catecholaminergic neurones. In this way dysfunction of the 26S-proteasome can be restricted to catecholaminergic neurones.

Alternatively, by linking expression of the Cre enzyme to the expression of calcium calmodulin dependant protein kinase IIα, expression of the Cre enzyme, and thus dysfunction of any floxed genes, is restricted to the anterior forebrain. In this way dysfunction of the 26S-proteasome can be restricted to the anterior forebrain.

Transgenic mice according to the invention can be produced in just a few weeks from the floxed colony of mice.

According to another aspect the invention provides a transgenic animal comprising one or more 26S proteasome associated ATPase genes which are floxed, at least in part, and means to cause expression of the Cre enzyme in some or all cells in the animal, wherein a dysfunction of the 26S-proteasome is observed in cells which express the Cre enzyme.

Preferably the ATPase which is floxed is an ATPase of the 19S regulator of the 26S-proteasome, more preferably it is the gene encoding the S4 ATPase which is floxed.

A transgenic animal according to this aspect of the invention may develop a neurodegenerative disease determined by where in the animal the 26S-proteasome dysfunction occurs.

All the preferred features described in relation to the first aspect of the invention can be applied to this aspect of the invention.

According to another aspect, the present invention provides a method of producing a transgenic non-human animal according to the first or second aspect of the invention comprising the steps of:
i) providing a first group of non-human animals, said first group of non-human animals being homozygous for a loxP-flanked gene, or a loxP-flanked part of a gene, which in the presence of the Cre enzyme results in the dysfunction of the 26S-proteasome;
ii) providing a second group of non-human animals, said second group of non-human animals expressing the Cre enzyme in some or all cells;
iii) crossing said first group of non-human animals with said second group of non-human animals, thereby obtaining a transgenic non-human animal genetically modified to have a dysfunction in the 26S-proteasome in some or all cells.

The first and second groups of animals are preferably mice.

Preferably the floxed (loxP flanked) gene is an ATPase gene, wherein the ATPase gene encodes an ATPase which forms part of the 19S regulator of the 26S-proteasome. Preferably the ATPase is the S4 ATPase. Preferably, in the absence of Cre recombinase, the loxP sequences in the floxed gene do not affect gene expression and/or function. The loxP sites may be within introns in an ATPase gene.

Preferably, the Cre enzyme is expressed in only some cells in the transgenic animal. Preferably expression of the Cre enzyme is linked to tyrosine hydroxylase expression, thus expression of the Cre enzyme is restricted to the catecholaminergic neurones. In this way dysfunction of the 26S-proteasome can be restricted to catecholaminergic neurones.

Alternatively, by linking expression of the Cre enzyme to the expression of calcium calmodulin dependant protein kinase IIα, expression of the Cre enzyme is restricted to the anterior forebrain. In this way dysfunction of the 26S-proteasome can be restricted to the anterior forebrain.

The skilled man will appreciate that all preferred features of the invention relating to a transgenic animal can be applied to all the methods of the invention.

A method of screening for compounds for treating a neurodegenerative disease, such as Parkinson's, dementia with Lewy bodies or Alzheimer's disease in which concomitant Lewy body disease is frequently present, comprising the steps of

i) providing a transgenic non-human animal as described above;
ii) providing a chemical to be tested;
iii) exposing the transgenic non-human animal to said compound to be tested;
iv) determining whether said chemical compound has properties which could be used to treat a neurodegenerative disease, such as Parkinson's, dementia with Lewy bodies or Alzheimer's disease.

According to another aspect, the invention provides a method for investigating whether a chemical compound could be useful for treating a neurodegenerative disease, such as Parkinson's, dementia with Lewy bodies or Alzheimer's disease in which concomitant Lewy body disease is frequently present, comprising the steps of

i) providing a transgenic non-human animal as described above;
ii) providing a chemical to be tested;
iii) exposing the transgenic non-human animal to said compound to be tested;
iv) determining whether said chemical compound has properties which could be used to treat a neurodegenerative disease, such as Parkinson's, dementia with Lewy bodies or Alzheimer's disease.

The properties the chemical compound could have include an ability to influence locomotion or memory in the transgenic non-human animals. The method may include evaluating the locomotor activity of the animal, and/or evaluating the effect on other symptoms diagnostic of Parkinson's disease and/or dementia with Lewy bodies and/or Alzheimer's disease.

According to a yet further aspect, the present invention provides the use of a compound identified according to any of the two preceding methods in the preparation of a pharmaceutical composition for the treatment of a neurodegenerative disease, such as Parkinson's disease and/or dementia with Lewy bodies and/or Alzheimer's disease.

According to a another aspect, the present invention provides a method for the treatment of a neurodegenerative disease, such as Parkinson's disease and/or dementia with Lewy bodies and/or Alzheimer's disease, comprising administering a compound identified according to a method of the invention,

Preferred embodiments of the invention will now be described merely by way of example with reference to the accompanying drawings in which:

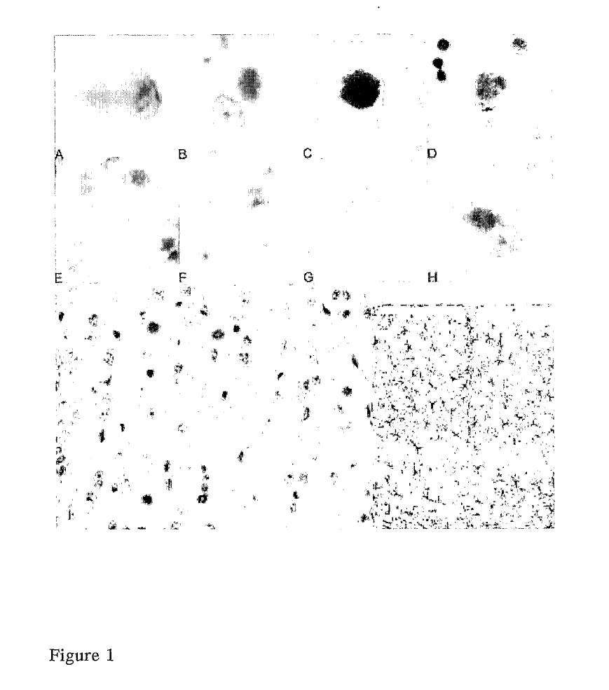

FIG. 1—illustrates cortical Lewy bodies in mice with conditionally-deleted S4 ATPase genes using histology and immunohistochemistry techniques. Cortical Lewy bodies (FIGS. 1E-H) identical to human cortical Lewy bodies (FIGS. 1A-1D) containing p62 (FIGS. 1B and 1F), α-synuclein (FIGS. 1C, 1G and 1I), and ubiquitin (FIGS. 1D and 1H) are shown to be extensive in the cortex by four weeks post-partum. At low magnification (×200) abundant synuclein-positive Lewy bodies can be seen in the cortex (FIG. 1I) together with extensive gliosis (FIG. 1J);

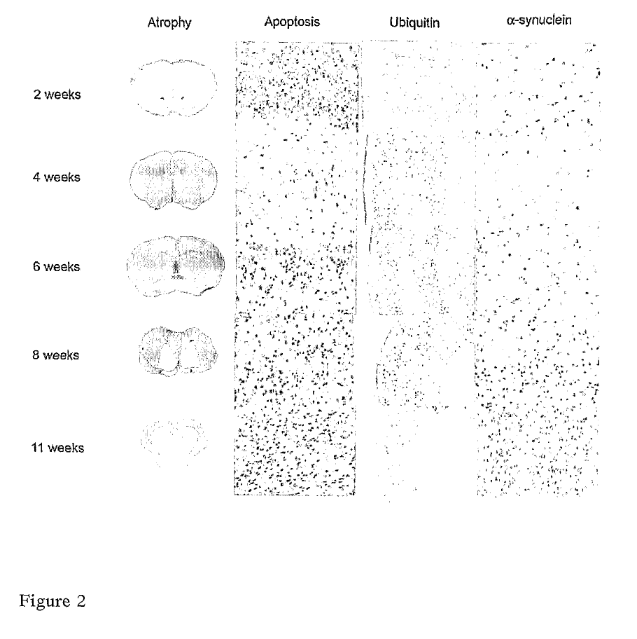

FIG. 2—shows the time course of brain atrophy and apoptosis, and ubiquitin and α-synuclein accumulation in Lewy bodies in mice in which the S4 ATPase gene has been conditionally-deleted. Brain atrophy accompanied by the formation of ubiquitin and α-synuclein-positive Lewy bodies takes place over a period of approximately 11 weeks;

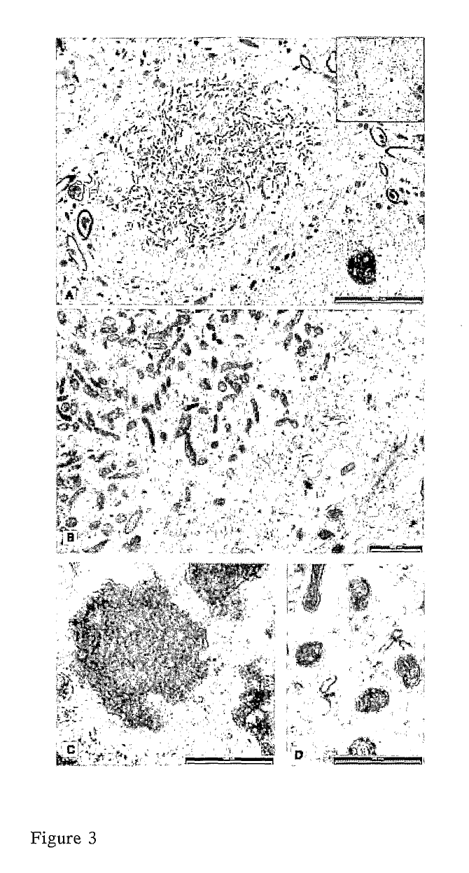

FIG. 3—illustrates the ultrastructure of Lewy bodies. Analysis of the ultrastructure of cortical Lewy bodies shows the paranuclear location (inset) of Lewy bodies (FIG. 3A), the periphery of a Lewy body with numerous mitochondria and vesicular structures (FIG. 3B), the centre of a Lewy body showing double-membraned dense bodies (FIG. 3C) and fine filaments and mitochondria (FIG. 3D);

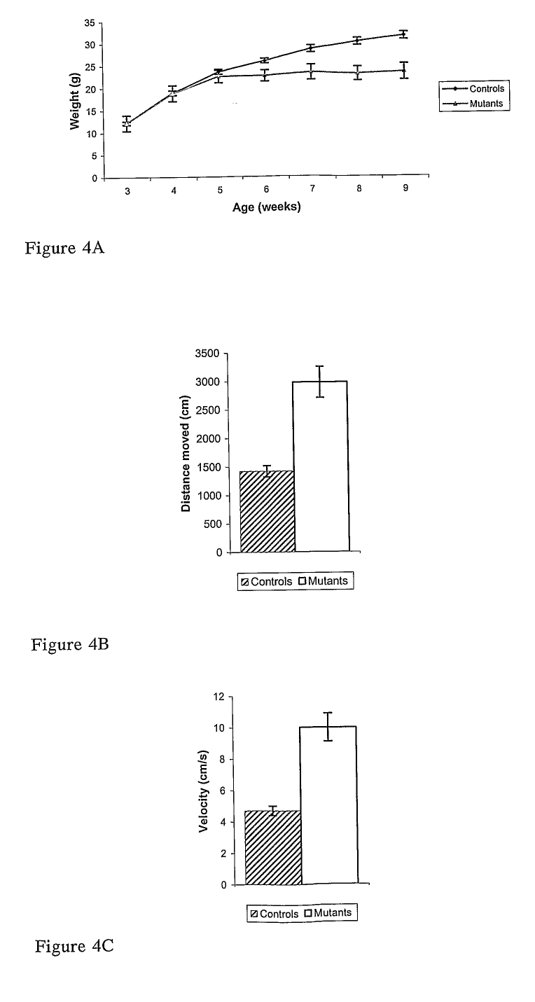

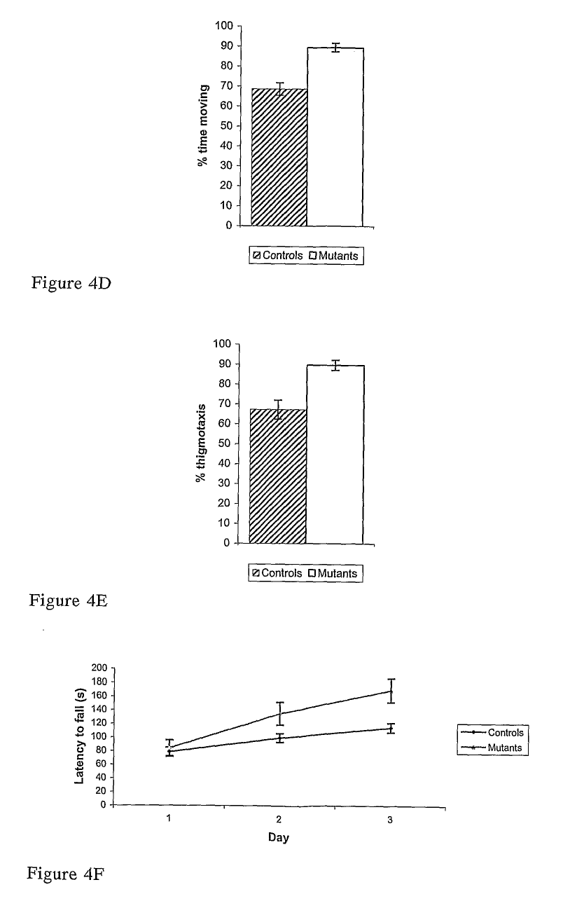

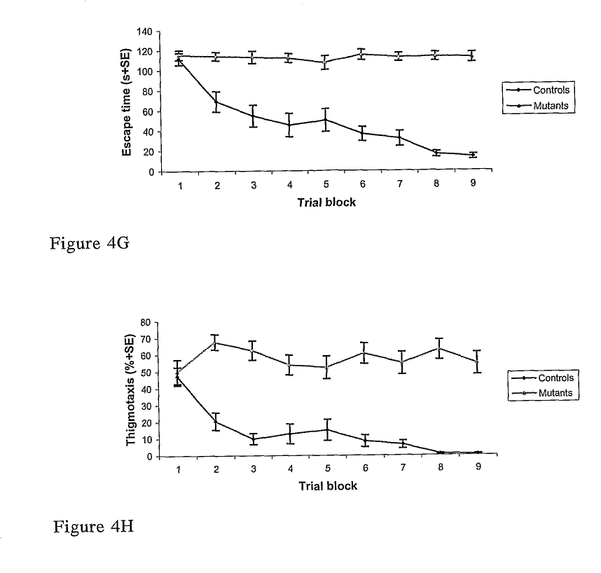

FIG. 4—illustrates behavioural and memory dysfunctions in mice in which the S4 ATPase gene has been conditionally-deleted;

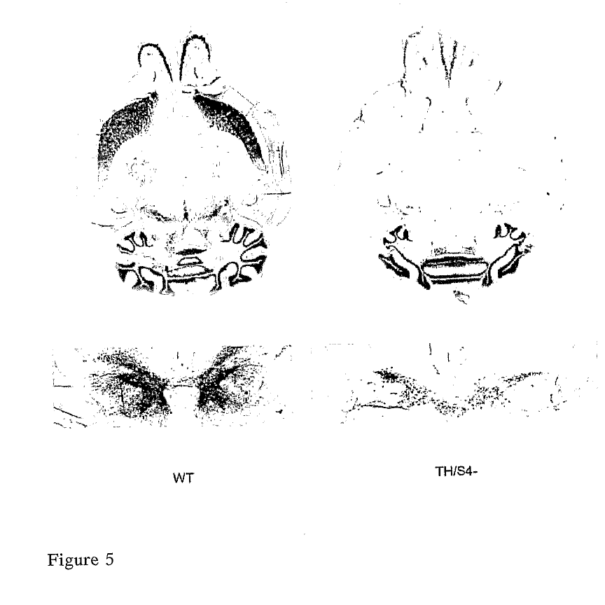

FIG. 5—illustrates the ablation of tyrosine hydroxylase-positive catecholaminergic neurones in mice in which the S4 ATPase gene has been conditionally-deleted. FIG. 5 shows that conditional-deletion of the S4 ATPase gene causes extensive loss of TH-positive neurones in the basal ganglia (caudate and putamen), substantia nigra and olfactory bulb;

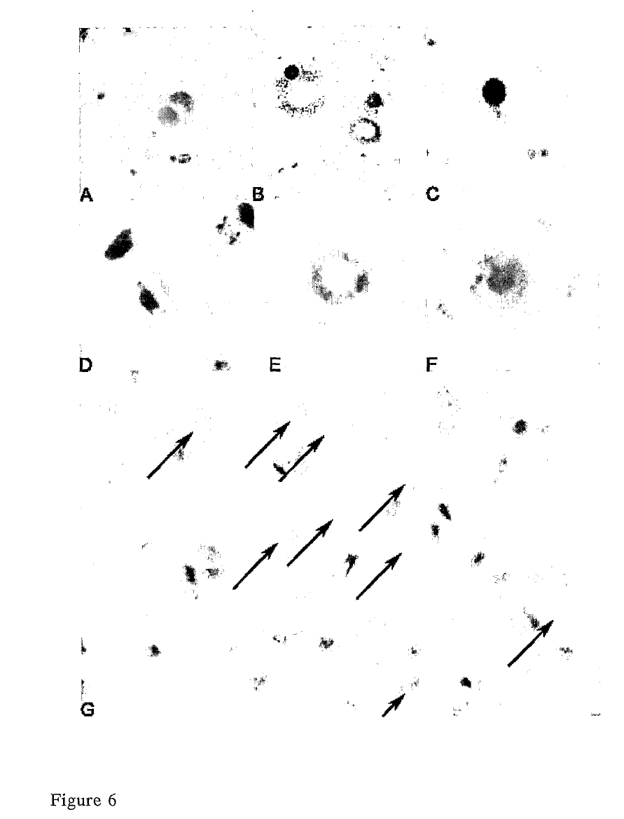

FIG. 6—shows that the conditional-deletion of the S4 ATPase gene in dopaminergic neurones generates Lewy bodies. Haemotoxylin/eosin stained Lewy bodies in the mouse substantia nigra (FIG. 6D) contain a-synuclein (FIG. 6E) and ubiquitylated proteins (FIG. 6F) as in human nigral Lewy bodies (FIG. 6A to 6C). The arrows indicate extensive Lewy bodies in the substantia nigra (FIG. 6G);

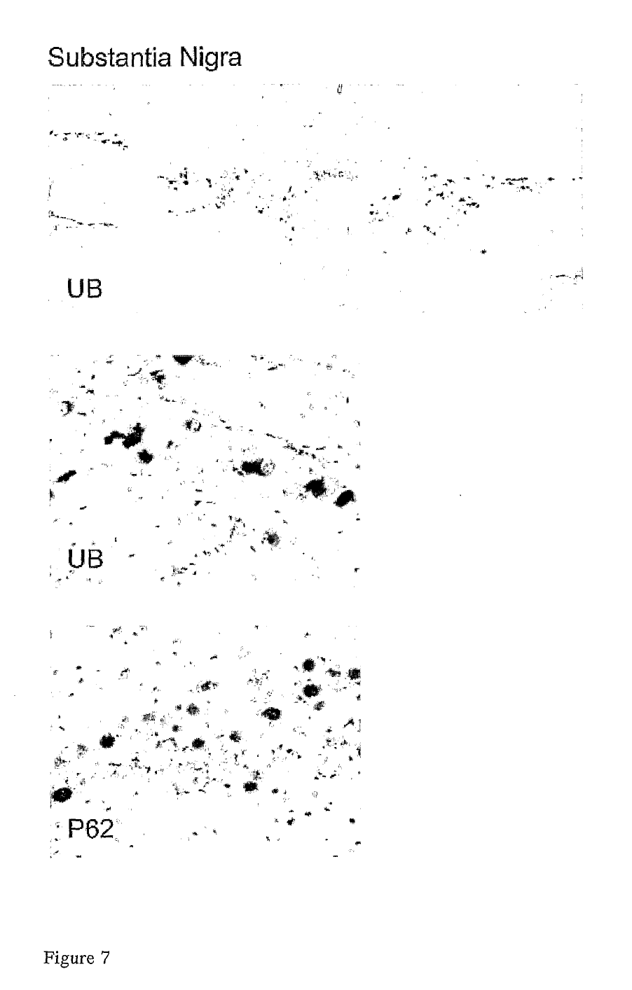

FIG. 7—shows that the substantia nigra of mice with conditional-deletion of the S4 ATPase gene contains ubiquitinylated proteins and p62. Low power magnification (×200) reveals that surviving nigral neurones contain diffuse immunoreactivity in neuronal cytoplasm for p62 including enhanced immunostaining in Lewy bodies. The pattern of p62 immunostaining mirrors ubiquitin immunostaining;

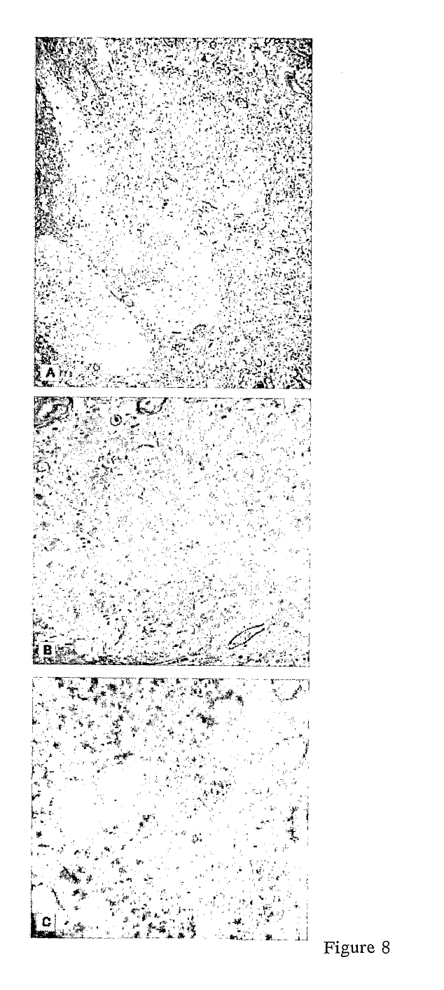

FIG. 8—illustrates Lewy body ultrastructure. Three adjacent neurones in the substantia nigra are illustrated containing juxtanuclear Lewy bodies (FIG. 8A), Lewy body containing mitochondria and peripheral electron-dense autophagolysosomes (FIG. 8B), the central composition of the nigral Lewy body is composed of granulovesicular material (FIG. 8C) corresponding to the later stage of human homogeneous Lewy bodies.

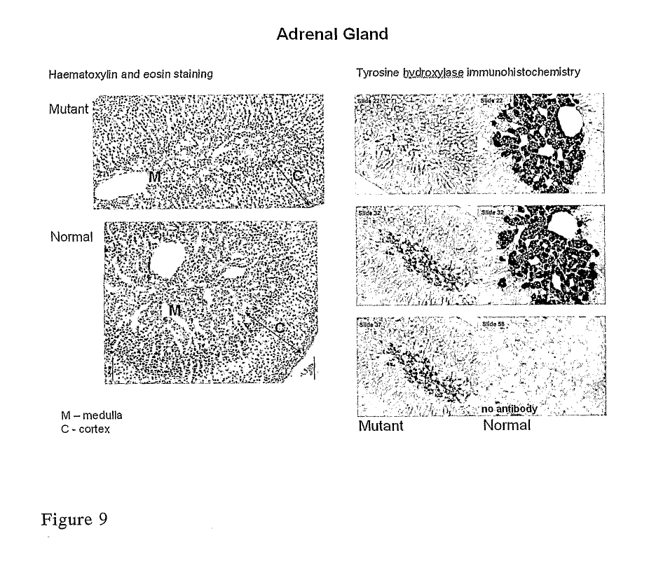

FIG. 9—shows the histology and TH-immunoreactivity of the adrenal medulla in mice in which the S4 ATPase gene has been conditionally-deleted in catechol aminergic neurones. Haematoxylin/eosin staining shows almost complete ablation of the adrenal medulla (left panels) as corroborated by tyrosine hydroxylase immunostaining (three right panels). (C) adrenal cortex (M) adrenal medulla;

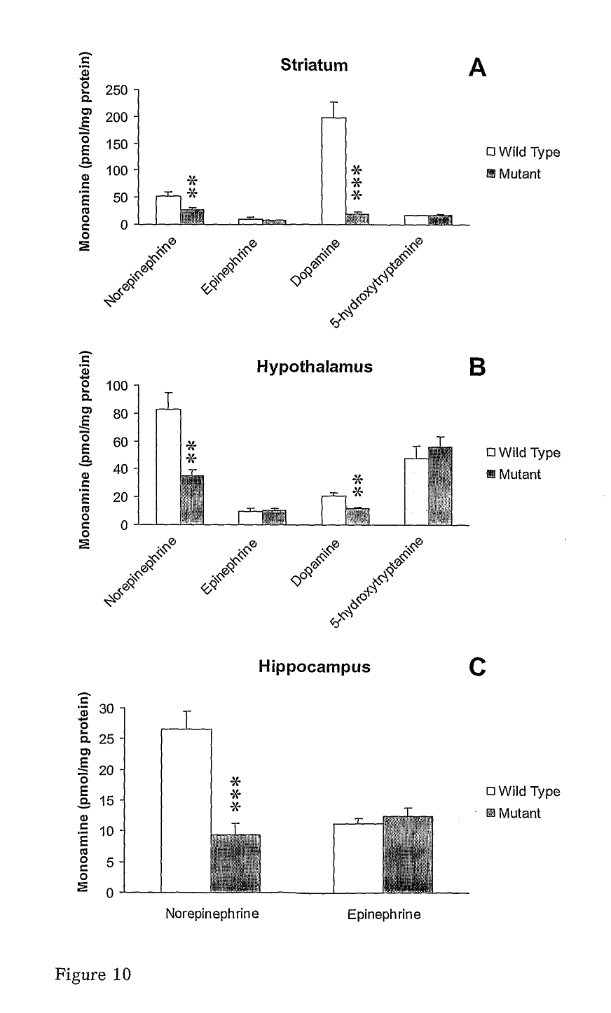

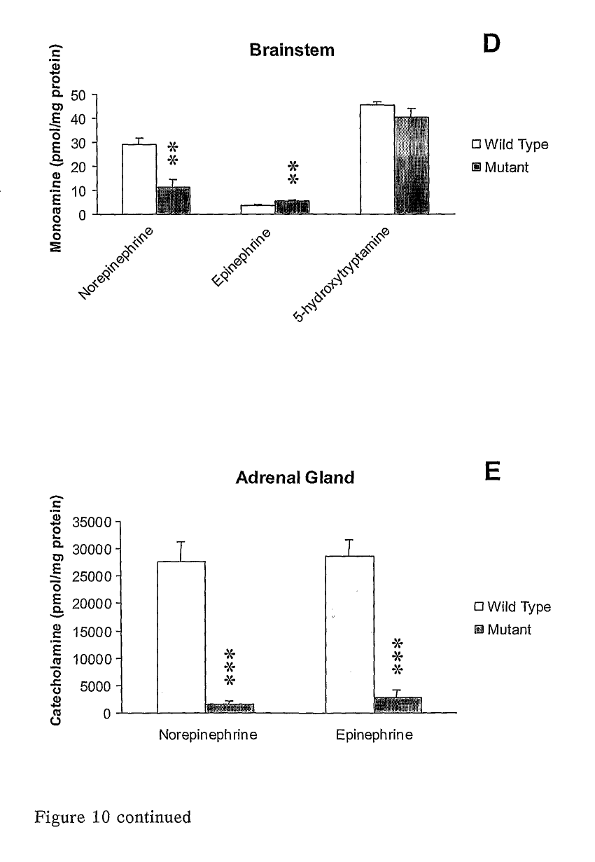

FIG. 10—shows that catecholamine but not indolamine levels are selectively reduced in the brain and adrenal medulla of mice with tyrosine hydroxylase-Cre directed conditional-deletion of the S4 ATPase gene. Tissue levels (pmol/mg protein, mean±s.e.m) of norepinephrine, epinephrine, dopamine and 5-hydroxytryptamine in the striatum (A) hypothalamus (B) hippocampus (C) brainstem (D) and adrenal gland (E) in n=5 wild type and n=7 S4 were measured by HPLC with electrochemical detection. **P<0.01 and ***P<0.001 from wild type mice. Student's unpaired t-test.

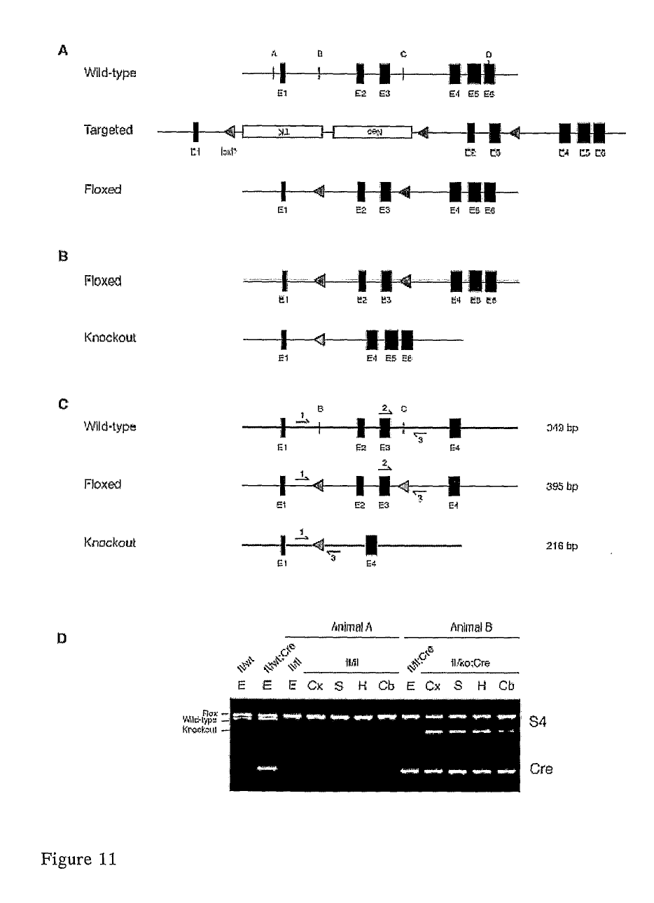

FIG. 11—illustrates the DNA targeting strategy used to produce transgenic mice in which the S4 ATPase gene is inactivated in the anterior fore brain. S4 ATPase genomic DNA between EcoRI and BamHI was cloned into pBluescriptSK for the targeting construct. A loxP-flanked selection cassette in reverse orientation and a loxP site were cloned into the HindIII and NcoI sites in introns 1 and 3 of the S4 ATPase gene respectively. The targeted allele was generated by homologous recombination in mouse 129/Sv embryonic stem (ES) cells. Transient expression of Cre recombinase in vitro excised the selection cassette. PCR amplification identified correctly targeted ES cell clones. (FIG. 11B). Floxed S4 ATPase mice were crossed with cell type-specific Cre-expressing mice leading to deletion of exons 2 and 3 (knockout), thus conditional inactivation of the S4 ATPase gene. (FIG. 11C)

The following primers were used for genotyping of the S4 allele:

(1) 5′CAGAAATACAGCCAGTGACC;
(2) 5′TACGAACCTCCTGTCCCAAC;
(3) 5′CTGGAACTCAGTGGATTGAG.

Primers used for the Cre recombinase were:

5′CGTACTGACGGTGGGAGAATG;
and
5′CCAGCTTGCATGATCTCCGG.

The PCR protocol used was: 3 min 94° C., 35 cycles (45 sec 94° C., 45 sec 60° C., 45 sec 72° C.), 3 min 94° C. (FIG. 11D) PCR amplification was performed on genomic DNA obtained from the ear, E, and the brain (Cx, cortex; S, striatum; H, hippocampus; Cb, cerebellum). The results of the PCR analysis indicate that inactivation of the S4 ATPase is restricted to the brain of S4f1/f1; CamKIIcre (mutant) animals.

### EXAMPLE 1

### Transgenic Mouse Model for Dementia with Lewy Bodies

This example demonstrates that the conditional-deletion of the proteasomal S4 ATPase gene in the anterior forebrain of mice causes the pathognomonic features of dementia with Lewy bodies accompanied by cognitive deficits.

The term “conditional-deletion” is used to refer to the deletion or inversion of all or part of a gene such that the gene is inactivated, the deletion/inversion is seen in only some cells of the body and is thus referred to as conditional

Mice homozygous for the “floxed” S4 ATPase gene were generated by introducing loxP sites into introns 1 and 3 of the S4 ATPase gene. The subsequent action of Cre-recombinase would prevent the synthesis of the S4 ATPase protein (FIG. 11). Spatial control of the deletion of the gene in the brain of mice with a floxed S4 ATPase gene was achieved by crossing the homozygous floxed mice with deletor mice expressing a CaMKIIα-Cre-recombinase. The resulting mice displayed deletion (inactivation) of the S4 ATPase gene in calcium calmodulin-dependent kinase IIα Cre-recombinase expressing neurones of the anterior forebrain only.

The CaMKIIα gene is expressed post-mitotically in the forebrain of mice, in particular it is expressed in the hippocampal pyramidal neurones, neocortex, striatum and amygdala (Tsien, J. Z. et al. Cell (1996) 87, 1317-26 and Minichiello, L. et al. Neuron (1999) 24, 401-14). The deletion of the S4 ATPase gene in the mouse forebrain causes the occurrence of numerous cortical Lewy bodies containing p62, α-synuclein, and ubiquitylated proteins (FIGS. 1E to 1I) accompanied by extensive astrocytic gliosis (FIG. 1J). Cortical Lewy bodies were observed by 28 days post-partum. The inclusions in the S4-conditional-deleted mouse brain are similar to those seen in the brains of patients succumbing to dementia with Lewy bodies (FIGS. 1A to 1D). Electron microscopy confirmed that the cortical Lewy bodies are juxtanuclear structures (inset: FIG. 3A) containing abundant mitochondria and filaments together with vesicular structures (FIG. 3B). The latter include double-membraned autophagosomes (FIG. 3C) and peripheral electron dense lysosomes (FIG. 3A) as well as surrounding membrane-bound vesicular structures suggestive of ER (FIG. 3B). The central composition of the cortical Lewy bodies were composed of mitochondria, fine filaments and vesicular structures (FIG. 3D) reminiscent of the description of homogeneous human Lewy bodies. The abundance of mitochondria in Lewy bodies after conditional-deletion of the S4 ATPase gene serves to emphasise the relationship between 26S proteasomes and mitochondria in Lewy body formation.

Extensive histological evidence for time-dependent cortical atrophy was observed, as shown by increased ventricle size (FIG. 2: left column) and apoptosis (FIG. 2: second column) to accompany ubiquitylated protein deposits (FIG. 2: third column) and synuclein-positive Lewy bodies (FIG. 2: left column) in the forebrain. The atrophy resulted in a reduction in the brain weight of mice with conditional-deletion of the S4 ATPase gene (not shown). In contrast, neuronal loss has generally not been seen in amyloid-generating transgenics and only in mutant-tau animals after many months (Gowan, E. et al. Trends in Genetics (2006) 22, 281-289). The occurrence of α-synuclein and ubiquitin-positive Lewy bodies together with extensive apoptosis peaked at approximately 6 weeks, as underscored by the loss of forebrain weight. By 11 weeks the proportion of neurones with Lewy pathology and neuritic change was smaller as might be expected with the dramatic previous reduction in neuronal number in afflicted regions of the brain. The accumulation of ubiquitylated proteins in the cortex preceded Lewy body pathology and was detectable at 2 weeks with a peak at approximately 4 weeks in the cortex (FIG. 2).

RT-PCR on RNA extracted from pyramidal cells obtained from the hippocampal formation by laser capture microdissection shows no S4 ATPase gene expression in the S4 ATPase gene deleted mice

Behavioural and memory analyses showed that after six weeks there was a statistically significant change in the performance of genetically modified animals (FIG. 4). The animals were also unkempt and did not use the bed-making materials. These cognitive abnormalities and behavioural changes have human parallels in dementia with Lewy bodies where visuospatial and attentional deficits are cognitive core symptoms (Guidi, M. et al. Journal of Neurological Sciences (2006) in press and Aarsland, D. et al. Archives of Neurology (2006) 60, 387-392).

No significant differences in body weight was observed during the initial growth phase of control and mice with a conditional-deletion of the S4 ATPase gene, However, from 5 weeks old mice with a conditional-deletion of the S4 ATPase gene did not gain weight like the control mice (FIG. 4A, P<0.05 from 8 weeks of age, t-test). Observations of general appearance and behaviour suggested that changes were apparent in mice with a conditional-deletion of the S4 ATPase gene from around 6 weeks of age. The mice with a conditional-deletion of the S4 ATPase gene became progressively unkempt with particularly piloerection on the nose and a hunched posture. These mice did not exhibit normal nesting behaviour. At 6 weeks, mice with a conditional-deletion of the S4 ATPase gene were significantly more active and exhibited increased anxiety in an open field arena compared to controls (FIG. 4B-E, P<0.01, t-test). This motor activity and emotionality was similar at 8 weeks of age (data not shown). In the accelerating rotarod test, control and mice with a conditional-deletion of the S4 ATPase gene improved over the 3 days, but mice with a conditional-deletion of the S4 ATPase gene showed significantly increased locomotor performance compared to that of control mice from the second day (FIG. 4F, day 2 P=0.05, day 3 P<0.01, t-test). Analysis of stride length demonstrated that this was not statistically different between mice with a conditional-deletion of the S4 ATPase gene and control mice (data not shown, P<0.01, t-test). Mice with a conditional-deletion of the S4 ATPase gene did not learn to locate a submerged platform in the Morris water maze task, suggesting a severe deficit in spatial learning (FIG. 4G). This cannot be explained by aberrant motor behaviour because both genotypes spent the majority of the task moving (data not shown, controls 95%±0.94, mutants 85%±1.48), although mice with a conditional-deletion of the S4 ATPase gene exhibited a significantly reduced swim speed compared to controls (data not shown, controls 24.23 cm/s±0.6, mice with a conditional-deletion of the S4 ATPase gene 10.74 cm/s±0.84). Mice with a conditional-deletion of the S4 ATPase gene exhibited persistent thigmotaxis during the task (FIG. 4H).

The combined data demonstrates that conditional genetic disruption of the 26S-proteasome in mice causes the neuropathological and behavioural features of dementia with Lewy bodies.

The prevalence of dementia with Lewy bodies in Parkinson's disease may be as high as 80% (McKeith et al. Lancet Neurology (2004) 3, 19-28). It is now quite clear that a subgroup of patients with dementia have a dominant pathology of Lewy bodies in the cortex and other brain regions. This is the subgroup of patients with the distinct clinical syndrome of dementia with Lewy bodies. The extent of neuronal loss and severity of presumably irreversible cortical damage may be less in patients with Lewy body pathology than in Alzheimer's disease. Therefore even when diagnosed at an advanced clinical stage a better window of therapeutic intervention may be present in patients with dementia with Lewy bodies than in Alzheimer's disease e.g. for treatment with acetyl cholinesterase inhibitors. It is now recognised that a subgroup of patients with Alzheimer's disease also have substantial brain Lewy bodies. The extent of Lewy body involvement varies and there may be further subtypes of disease e.g. with Lewy bodies in the amygdala only.

The development of a mouse model for dementia with Lewy bodies offers a unique model to investigate new strategies to prevent neuronal death in relation to Lewy body pathology in the human brain.

### Methods

Floxed S4 mice. Utilising homologous recombination, a loxP-flanked neomycin-thymidine kinase selection cassette and a loxP site were inserted into introns 1 and 3 respectively of the S4 gene in mouse 129/Sv embryonic stem (ES) cells. The selection cassette was excised by transient expression of Cre recombinase in vitro (FIG. 11A). PCR amplification identified correctly targeted ES cell clones. Male chimeric mice were generated using aggregation (Tanaka et al. Meths. Mol. Biol. (2001) 158, 135-154) with CD1-derived morulae and bred with CD1 females to establish homozygous floxed S4 mice (S4^{f1/f1}). More detail on the generation of the floxed mice is, given in the legend to FIG. 11.

Cell type-specific inactivation of S4. The S4^{f1/f1} mice were crossed with CamKIIα^{cre} mice (Lindeberg, J. et al. Journal of Neuroscience Research (2002) 68, 248-253) generating S4^{f1/wt}; CamKIIα^{cre} and S4^{f1/wt}; CamKIIα^{wt} mice. Female S4^{f1/wt}; CamKIIα^{cre} mice were mated with S4^{f1/wt} males to produce mice in which S4 was specifically inactivated in calcium-calmodulin-dependent protein kinase IIα-expressing cells (S4^{f1/fl}; CamKIIα^{cre} or S4^{f1/ko}; CamKIIα^{cre}) and control mice (e.g. S4^{f1/fl}; CamKIIα^{wt} or S4^{f1/wt}; CamKIIα^{cre}). Further information in the legend to FIG. 11

Genotyping. PCR amplification of ear biopsy DNA was used for genotyping (FIGS. 11C and 11D).

Histology. For light microscopy, mice were perfusion fixed with 4% paraformaldehyde in 0.1 M phosphate buffer (pH 7.4), brains dissected, embedded in paraffin wax and sectioned (5 μm) according to standard protocols. General morphological examination used haematoxylin (Harris) and eosin staining. Immunostaining was performed as directed in Vector Laboratories M.O.M. Immunodetection or Vectastain Elite rabbit IgG ABC kits. Antigen retrieval used microwave treatment in 0.01 M citrate buffer containing 0.05% Tween-20 (pH 6) or 1 mM EDTA followed by formic acid treatment. Primary antibody incubation was for 1 h at room temperature: 1:5000 dilution for ubiquitin (Chemicon); 1:5 dilution for synuclein (Vector Laboratories) or 1:10 000 dilution for synuclein; 1:1000 p62 (BD Transduction Laboratories). DAPI (1 μg/mL) was used to visualise cell nuclei. Analysis used a Leica LSM 500 microscope and packaged software.

When cryosections were needed, brains were dissected and frozen in pre-cooled isopentane over liquid nitrogen. 3.2% paraformaldehyde, 0.2% glutaraldehyde, 1% sucrose, 3 mM CaCl₂ in 0.1 M sodium cacodylate buffer was used for electron microscopic analysis.

Western blot analysis. Tissues were homogenised in ice-cold PBS and protein extracts (50 μg) separated by SDS-polyacrylamide gel electrophoresis, transferred to nitrocellulose and immunoblotted with antibodies to the 20S core of the 26S proteasome, to the S4 ATPase and to the S10b ATPase.

### Behavioural Analyses

Rotarod. Motor activity of the mice was assessed using an accelerating rotarod (4 to 40 rpm in 300 s, LE 8200). 300 s was recorded for animals walking over this time. On 3 consecutive days mice were given 3 trials (inter-trial time approx 30 min). Day 1 data was not included in statistical analyses.

Open Field. Each mouse was monitored for 5 min in an open field arena (350 mm×300 mm×300 mm; 3 walls transparent, 1 wall and floor dark grey). Activity in the open field was recorded and quantitated by a computer operated animal activity system.

Morris Water Maze. Mice were placed in an opaque pool (approximately 25° C.) and given 120 s to locate a submerged platform. Acquisition was performed over 3 consecutive days. Each day mice undertook 3 blocks of 2 trials lasting 120 seconds each with an interblock interval of around 1 hour (18 trials total over 3 days). Two compass points located on the pool were used. The time taken to reach the platform (latency to escape) was recorded for each trial. The 120 seconds probe trial in which the platform was removed was on day 4 from a start position opposite the original platform location.

### EXAMPLE 2

### Transgenic Mouse Model for Parkinson's Disease

This example shows that conditional-deletion of the proteasomal S4 ATPase gene in catecholaminergic neurones, including dopaminergic neurones, in the brains of mice causes the hallmark features of Parkinson's disease. The S4 gene was chosen for this study because of the key role of the S4 ATPase in the mechanism of action of the 26S-proteasome (Rubin et al. EMBO J (1998) 17, 4909-4919).

Mice homozygous for the “floxed” S4 ATPase gene were generated by introduction of loxP sites in introns 1 and 3 so that the subsequent action of Cre-recombinase would prevent the synthesis of the S4 ATPase protein.

Spatial control of the deletion of the gene in the brain was achieved by crossing homozygous S4 floxed mice with deletor mice expressing a tyrosine hydroxylase-internal ribosome entry site-Cre recombinase cassette (TH-IRES-Cre) to confine ablation of the gene to catecholaminergic neurones. The deletion of the gene in the nigrostriatal pathway was demonstrated in the same way as for the gene in the forebrain with the calcium calmodulin kinase cassette (FIG. 11). The deletion of the S4 gene was demonstrated by RT-PCR of RNA from neurones laser-microdissected from tyrosine hydroxylase positive remaining neurones in the substantia nigra and adrenal medulla (not shown).

Expression of endogenous tyrosine hydroxylase began at around E10.5 days (not shown). By three weeks post-partum tyrosine hydroxylase (TH) immunostaining showed substantial loss of TH-positive neurones in the basal ganglia (FIG. 5: top left and right panels), extensive depletion of TH-positive neurones in the substantia nigra (FIG. 5: lower panels) and loss of immunostaining in the olfactory bulb (FIG. 5: top left and right panels). These hallmark neuropathological features of Parkinson's disease were seen within 21 days of gene deletion (FIG. 6). Histological examination shows that deletion of the S4 gene causes numerous haematoxylin/eosin positive Lewy bodies (FIG. 6D) adjacent to neuronal nuclei in remaining neurones in the substantia nigra. The Lewy bodies contain α-synuclein (FIG. 6E) and ubiquitylated proteins (FIG. 6F) as seen in human Lewy bodies (FIGS. 6A to 6C). Lewy bodies were extensively distributed in the substantia nigra (FIG. 6G). Surviving neurones in the substantia nigra showed diffuse immunoreactivity in neuronal cytoplasm for the p62 protein including enhanced immunostaining in Lewy bodies. The pattern of p62 immunostaining mirrored ubiquitin immunostaining (FIG. 7) and was similar to that seen for p62 immunoreactivity in human Lewy bodies. At the ultrastructural level juxtanuclear Lewy bodies (FIG. 8A) contain numerous mitochondria together with granulovesicular material. Confocal microscopy showed perinuclear α-synuclein and ubiquitin immunostaining with a focused coincidence of immunostaining associated with the microtubule organising centre as demonstrated by immunostaining for α-tubulin (not shown).

Quite remarkably, these molecular neuropathological features are the major characteristics of Parkinson's disease. As was expected from the exploitation of the tyrosine hydroxylase gene locus to control Cre-recombinase activity, autonomic dysfunction, including ablation of catecholaminergic neurones in peripheral tissues (e.g. liver and adipose tissue, not shown) together with almost complete elimination of the adrenal medulla, occurred in the animals by 21 days after commencement of deletion of the S4 gene (FIG. 9).

The neuropathological features were accompanied by catecholaminergic deficits in the brain, which included a statistically significant large decrease in dopamine and norepinephrine in the striatum (FIG. 10A), hypothalamus (FIG. 10B) hippocampus (FIG. 10C) and brain stem (FIG. 10D) similar to those seen in Parkinson's disease. As expected elimination of TH-expressing neurones in the substantia nigra not only prevented dopamine biosynthesis but also downstream synthesis of norepinephrine and epinephrine. In the adrenal medulla, where norepinephrine and epinephrine are present in very large amounts (in comparison to levels in the selected brain regions), conditional S4 ATPase gene deletion caused an almost complete and comparable decrease in both monoamines (FIG. 10E). The reduction in norepinephrine is presumably so extensive that very little epinephrine can be produced because of substrate starvation for phenolethylamine-N-methyl transferase. In contrast, in some brain regions S4 gene deletion had a more dominant effect on dopamine and norepinephrine with relatively little effect on of the epinephrine. This can be explained by residual catecholaminergic neurons in the substantia nigra (FIGS. 5 and 7), neurones responsible for the production of epinephrine. It is particularly noteworthy that in the brainstem, hypothalamus and striatum S4 ATPase gene ablation had no effect on the levels of serotonin showing exclusive selectivity for TH containing mono aminergic neurones.

The neuropathological features of Parkinson's disease occurred progressively from initiation of expression of TH-IRES-Cre recombinase.

Electron microscopic assessment of the Lewy-like bodies in the substantia nigra (FIG. 8) revealed a substructure of amorphous protein aggregates surrounded by mitochondria and membranes including double-membraned autophagosomal structures.

In S4-deficient surviving neurones in the substantia nigra the formation of aggresomes and utilisation of the autophagic system are the only known options for the isolation and/or degradation of otherwise toxic proteins including the natively-unfolded aggregate-prone α-synuclein. The conditional-deletion of the S4 ATPase gene generated aggresomes as evaluated by current criteria and conditional S4 ATPase gene-deleted mice will provide a simplified platform in which to study aggresome formation and the molecular detail of autophagy in the brain or in any other tissue or cell type with the appropriate tissue or cell specific promoter-controlled Cre-recombinase.

The observations in the conditional-deleted S4 ATPase gene mice demonstrate the central importance of the 26S proteasome in dopaminergic neurones to avoid the onset of Parkinsonian pathology and show the consequences of deletion of the gene for disease occurrence. The features of the disease can be modelled in 21 days: this observation has considerable implications for the human disorder in terms of understanding the length of time needed to generate Lewy bodies and accompanying Parkinsonian features in dopaminergic neurones.

To mimic the age-related motor dysfunctions of Parkinson's disease, a viral-Cre recombinase may be stereotactically injected into the substantia nigra of ageing animals bearing the floxed S4 ATPase gene. The age-related model will be invaluable for further understanding of Parkinson's disease and for the much-needed development of new therapies to combat the death of nigrostriatal neurones in the disorder.

### Methods

Floxed S4 mice. Utilising homologous recombination, a loxP-flanked neomycin-thymidine kinase selection cassette and a loxP site were inserted into introns 1 and 3 respectively of the S4 gene in mouse 129/Sv embryonic stem (ES) cells. The selection cassette was excised by transient expression of Cre recombinase in vitro (FIG. 11A). PCR amplification identified correctly targeted ES cell clones. Male chimeric mice were generated using aggregation (Tanaka et al. Meths. Mol. Biol. (2001) 158, 135-154) with CD1-derived morulae and bred with CD1 females to establish homozygous floxed S4 mice (S4^{f1/f1}). More detail on the generation of the foxed mice is given in the legend to FIG. 11.

Cell type-specific inactivation of S4. The S4^{f1/f1} mice were crossed with TH^{cre} mice (Lindeberg, J. et al. Genesis (2004) 40, 67-73) generating S4^{f1/wt}; Th^{cre} and S4^{f1/wt}; Th^{wt} mice. Male S4^{f1/wt}; Th^{cre} mice were bred with S4^{f1/f1} females to produce mice deficient in S4 specifically in tyrosine hydroxylase expressing cells (S4^{f1/f1}; Th^{cre} or S4^{f1/ko}; Th^{cre}) and control mice (e.g. S4^{f1/f1}; Th^{wt} or S4^{f1/wt}; Th^{cre}).

Genotyping. PCR amplification of ear biopsy DNA was used for genotyping (in a manner similar to FIGS. 11C and 11D).

Histology. For light microscopy, mice were perfusion fixed with 4% paraformaldehyde in 0.1 M phosphate buffer (pH 7.4), brains dissected, embedded in paraffin wax and sectioned (5 μm) according to standard protocols. General morphological examination used Haematoxylin (Harris) and Eosin staining. Immunostaining was performed as directed in Vector Laboratories M.O.M. Immunodetection or Vectastain Elite rabbit IgG ABC kits. Antigen retrieval used microwave treatment in 0.01 M citrate buffer containing 0.05% Tween-20 (pH 6) or 1 mM EDTA followed by formic acid treatment. Primary antibody incubation was for 1 h at room temperature: 1:5000 dilution for ubiquitin (Chemicon); 1:5 dilution for synuclein (Vector Laboratories) or 1:10 000 dilution for synuclein; 1:1000 p62 (BD Transduction Laboratories). DAPI (1 μg/mL) was used to visualise cell nuclei. Analysis used a Leica LSM 500 microscope and packaged software.

When cryosections were needed, brains were dissected and frozen in pre-cooled isopentane over liquid nitrogen. 3.2% paraformaldehyde, 0.2% glutaraldehyde, 1% sucrose, 3 mM CaCl₂ in 0.1 M sodium cacodylate buffer was used for electron microscopic analysis.

Western blot analysis. Tissues were homogenised in ice-cold PBS and protein extracts (50 μg) separated by SDS-polyacrylamide gel electrophoresis, transferred to nitrocellulose and immunoblotted with antibodies to the 20S core of the 26S proteasome, to the S4 ATPase and to the S10b ATPase.

Monoamine analysis by HPLC-ED. Selected brain regions were extracted and analysed for norepinephrine, epinephrine, dopamine and 5-hydroxytryptamine using HPLC with electrochemical detection. Individual brain tissue was extracted in 0.1M ice cold perchloric acid containing 0.02% (w/v) sodium metabisulphite (0.2 to 1 ml dependent on region) by sonication (10 s, MSE Soniprep) and the supernatant was collected following centrifugation (10 min, 4° C. at 17500 g, MSE Harrier 18/80). Resultant samples were analysed immediately following filtration through 0.45 μm filteres (Acrodisc, PALL) and kept on ice during the process.

For HPLC analysis samples were separated on a 150×4.6 mm Nucleosil HPLC column (C18 ODS, 5 μm Phenomenex) using a mobile phase comprising of 10% (v/v) methanol, 0.05M potassium dihydrogen phosphate, 0.1 mM EDTA, 2 mM octane sulphonic acid, pH 3.5, at a flow rate of 0.6 ml/min (Dionex P680). Detection was by an Antec (INTRO) electrochemical detector with a glassy carbon working electrode set at a potential of +0.8 V. Sample concentrations were calculated against standards using Galaxie integration software (Varian).

1. A transgenic non-human animal genetically modified to have a dysfunctional 26S-proteasome in some or all cells.

2. A transgenic non-human animal according to claim 1 wherein the non-human animal is a mammal.

3. A transgenic non-human animal according to claim 1 wherein the mammal is a mouse or a rat.

4. A transgenic non-human animal according to claim 1 wherein dysfunction of the 26S-proteasome arises due to a dysfunction in one or more of the ATPases of the 19S regulator of the 26S-proteasome.

5. A transgenic non-human animal according to claim 4 wherein one or more of the ATPases of the 19S regulator of the 26S-proteasome are genetically modified.

6. A transgenic non human animal according to claim 5 wherein the one or more ATPases, which are genetically modified to be dysfunctional, are selected from the 26S-proteasome ATPases S4, S6a, S6b, S7, S8 and S10b.

7. (canceled)

8. A transgenic non-human animal according to claim 1 wherein the transgenic animal is modified such that there is a dysfunction in the 26S-proteasome in only some of the cells of the transgenic animal.

9. A transgenic non-human animal according to claim 8 wherein dysfunction of 26S-proteasome occurs in cells of the anterior forebrain or in catecholamine neurones.

10. (canceled)

11. A transgenic non-human animal according to claim 1 wherein dysfunction of the 26S-protesome is caused by using the Cre-loxP system.

12. A transgenic non-human animal according to claim 11 wherein one or more of the ATPases of the 26S-proteasome is floxed.

13. A transgenic non-human animal according to claim 12 wherein the Cre enzyme is expressed in some or all cell types of the animal.

14. A transgenic non-human animal according to claim 1 which exhibits phenotypic and/or neuropathological symptoms similar to those exhibited by individuals with a neurodegenerative disorder.

15. (canceled)

16. A transgenic non-human animal comprising one or more 26S proteasome associated ATPase genes which are floxed at least in part, and means to cause expression of the Cre enzyme in some or all cells in the animal, wherein in cells which express the Cre enzyme a dysfunction of the 26S proteasome is observed.

17. A transgenic non-human animal according to claim 16 wherein the one or more ATPase genes which are floxed encode one or more ATPases of the 19S regulator of the 26S-proteasome.

18. A method of producing a transgenic non-human animal according to claim 1 comprising the steps of:
i) providing a first group of non-human animals, said first group of non-human animals being homozygous for a loxP-flanked gene which in the presence of the Cre enzyme results in the dysfunction of the 26S-proteasome;
ii) providing a second group of non-human animals, said second group of non-human animals expressing the Cre enzyme in some or all cells;
iii) crossing said first group of non-human animals with said second group of non-human animals, thereby obtaining a transgenic non-human animal genetically modified to have a dysfunction in the 26S-proteasome in some or all cells.

19. A method according to claim 18 wherein the first and second groups of animals are mice.

20. A method according to claim 18 wherein the loxP flanked (floxed) gene is an ATPase gene encoding an ATPase which forms part of the 19S regulator of the 26S-proteasome.

21. A method according to claim 18 where the Cre enzyme is expressed in catecholaminergic neurones or in the anterior forebrain.

22. (canceled)

23. A method for investigating whether a chemical compound could be useful for treating a neurodegenerative disease, such as Parkinson's, dementia with Lewy bodies or Alzheimer's disease in which concomitant Lewy body disease is frequently present, comprising the steps of
i) providing a transgenic non-human animal according to claim 1;
ii) providing a chemical to be tested;
iii) exposing the transgenic non-human animal to said compound to be tested;
iv) determining whether said chemical compound has properties which could be used to treat a neurodegenerative disease, such as Parkinson's, dementia with Lewy bodies or Alzheimer's disease.

24. A method according to claim 23 also comprising the step of evaluating the locomotor activity of the animal, and/or evaluating the effect on other symptoms diagnostic of Parkinson's disease and/or dementia with Lewy bodies and/or Alzheimer's disease.

25. (canceled)

26. A method for the treatment of neurodegenerative diseases, such as Parkinson's and/or Dementia with Lewy bodies and/or Alzheimer's disease, comprising administering a compound identified according to the method of claim 23.

27. (canceled)

28. (canceled)


SEQUENCE LISTING (5 sequences)

SEQ ID NO: 1 - DNA, 20 bp, Artificial
  Primer
cagaaatacagccagtgacc

SEQ ID NO: 2 - DNA, 20 bp, Artificial
  Primer
tacgaacctcctgtcccaac

SEQ ID NO: 3 - DNA, 20 bp, Artificial
  Primer
ctggaactcagtggattgag

SEQ ID NO: 4 - DNA, 21 bp, Artificial
  Primer
cgtactgacggtgggagaatg

SEQ ID NO: 5 - DNA, 20 bp, Artificial
  Primer
ccagcttgcatgatctccgg